(19) United States
(12) Plant Patent Application Publication
(10) Pub. No.: US 2014/0075622 P1
(43) Pub. Date: Mar. 13, 2014

(54) ARTICHOKE PLANT NAMED 'SJ215'

(50) ;

(71) Applicant: Steve Jordan, Lompoc, CA (US)

(72) Inventor: Steve Jordan, Lompoc, CA (US)

(21) Appl. No.: 13/854,901

(22) Filed: Apr. 1, 2013

Related U.S. Application Data
(60) Provisional application No. 61/686,295, filed on Apr. 2, 2012.

(51) Int. Cl. A01H 5/00

(52) U.S. Cl. PLT/258

(57) ABSTRACT

A new cultivar of artichoke named ‘SJ215’ that is distinguishable by the color of the edible immature flower buds as red. This trait set ‘SJ215’ apart from all other existing varieties of Cynara known to the inventor.

### CROSS-REFERENCE TO RELATED APPLICATIONS

This is a Non-Provisional Plant Patent Application based upon Provisional Patent Application No. 61/686,295 as filed on Apr. 2, 2012, and claims priority under 35 U.S.C. 199(e).

### GENUS AND SPECIES

CYNARA scolymus

### VARIETY DENOMINATION

‘SJ215’

### STATEMENT REGARDING FEDERALLY SPONSORED RESEARCH OR DEVELOPMENT

None

### BACKGROUND AND SUMMARY OF THE INVENTION

The present invention relates to a new and distinct cultivar of artichoke plant known commonly as Globe artichoke a perennial herb that is grown as a food crop for the production of edible vegetable delicacies. The new invention is known botanically as CYNARA scolymus and will be referred to hereinafter by the cultivar name ‘SJ215’. Cynara is in the family Asteraceae, formerly known as Compositae.

An individual plant of Cynara scolymus ‘SJ215’ is comprised of main stem, lateral stems, leaves, and immature flower buds known as involucra. Each involucrum is made up of imbricate phyllaries also known as involucral bracts. Freshly harvested immature flower buds can be baked, grilled, boiled, or steamed, making the fleshy part of the bracts, the fleshy receptacle, and the uppermost part of the peduncle, edible. If not harvested, but permitted to mature, flower buds will develop into flower heads known as capitula.

The new Cynara variety named ‘SJ215’ was discovered as a natural somaclonal variant (sport) derived in a commercial crop in Lompoc, Calif. from the following parent: An individual Cynara scolymus ‘BCL 9-11’ (U.S. Plant Pat. No. 15,807).

The closest comparison plant is Cynara scolymus ‘BVC 8-11’ (PP 164299‘BCL 9-11’ (U.S. Plant Pat. No. 15,807). The color of the edible immature flower buds of Cynara scolymus ‘SJ215’ is red. By comparison, the color of the edible immature flower buds of the parent Cynara scolymus ‘BCL 9-11’ (U.S. Plant Pat. No. 15,807) is green.

The first asexual propagation of the new variety named ‘SJ215’ was conducted in the inventor's laboratory in Lompoc, Calif. in 2009. The method used was tissue culture. Since that time field propagation has been carried out using the method of division. Under careful observation, ‘SJ215’ has been determined uniform, stable and true to type in subsequent generations of asexual propagation.

### SUMMARY OF THE INVENTION

The following traits have been repeatedly observed and represent the distinguishing characteristics of the new Cynara cultivar named ‘SJ215’. These traits in combination distinguish ‘SJ215’ from all other existing varieties of Cynara known to the inventor. ‘SJ215’ has not been tested under all possible conditions and phenotypic differences may be observed with variations in environmental, climatic, and cultural conditions, however, without any variance in genotype.

1. The immature flower buds of ‘SJ215’ are red in color.

### BRIEF DESCRIPTION OF THE DRAWINGS

The accompanying color drawings illustrate the overall appearance of the new Cynara cultivar named ‘SJ215’ showing color as true as is reasonably possible to obtain in color reproductions of this type. Color in the drawings may differ from color values cited in the detailed botanical description, which accurately describe the actual color of the new variety ‘SJ215’.

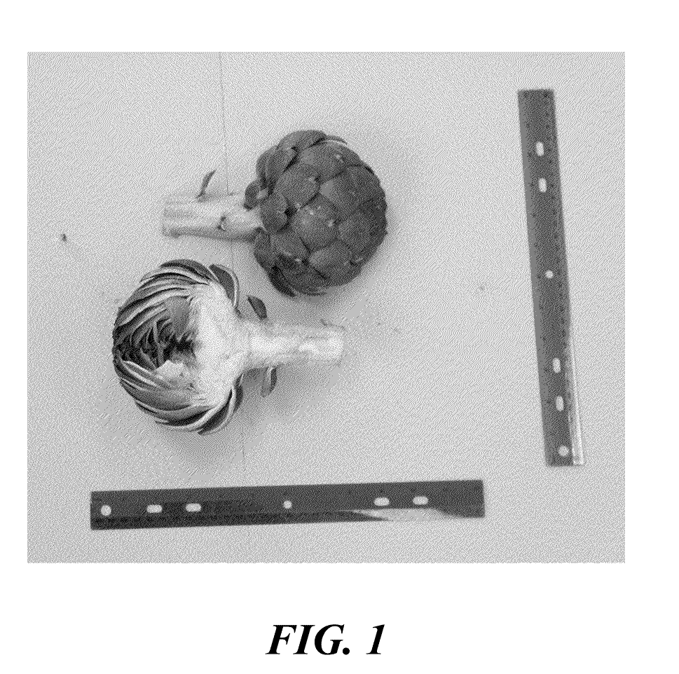

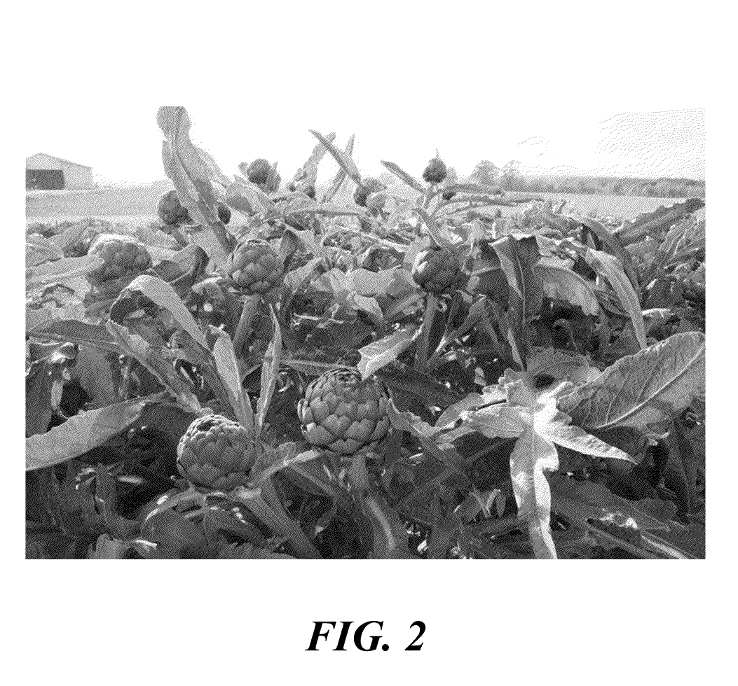

Drawing labeled FIG. 1 depicts immature flower buds of ‘SJ215’. Drawing labeled FIG. 2 depicts the whole artichoke plant. The drawings were made using conventional techniques and although immature flower bud and foliage color may appear different from actual color due to light reflectance, it is as accurate as possible by conventional photography.

### DETAILED BOTANICAL DESCRIPTION

The following is a detailed botanical description of the new Cynara cultivar named ‘SJ215’. Color determinations are in accordance with The 2001 Royal Horticultural Society Colour Chart of London, England, except where general color terms of ordinary dictionary significance are used. The following observations, measurements and values describe the new cultivar named ‘SJ215’ as grown in Lompoc, Calif. under conditions used in horticultural practice.

Lompoc, Calif. is located on the central coast in Santa Barbara County. Conditions in Lompoc vary with air temperatures ranging from 15.5° Centigrade to 26.6° Centigrade. The relative humidity is generally high. Prevailing winds are northwesterly and rainfall averages twelve inches per year. Growing requirements are similar to the species.
Botanical classification: CYNARA scolymus ‘SJ215’.
Family: Asteraceae (formerly Compositae).
Genus: CYNARA.
Species: Scolymus.
Denomination: ‘SJ215’.
Common name: Globe artichoke.
Commercial classification: Food crop.
Use: Production of edible vegetable delicacies.
Parentage: Cynara scolymus ‘SJ215’ is a natural somaclonal variant (sport) derived in a commercial crop in Lompoc, Calif. from the following parent: An individual Cynara scolymus ‘BCL 9-11’ (U.S. Plant Pat. No. 15,807)

1. A new and distinct cultivar of artichoke plant named ‘SJb 215’ as described and illustrated herein.
